(19) United States
(12) Patent Application Publication
(10) Pub. No.: US 2008/0004481 A1
(43) Pub. Date: Jan. 3, 2008

(54) APPARATUS AND METHOD FOR GUIDING INSERTION OF A MEDICAL TOOL

(76) Inventors: Jeffrey Bax, London (CA); Aaron Fenster, London (CA); Jacques Montreuil, London (CA); Lori Gardi, London (CA); David Martin Smith, London (CA)

Correspondence Address: BRUNET & CO. LTD., 10712 MELROSE DR., KOMOKA, ON, N0L-1R0, US

(21) Appl. No.: 11/427,121

(22) Filed: Jun. 28, 2006

(51) Int. Cl. A61M 36/00

(52) U.S. Cl. 600/7

(57) ABSTRACT

An apparatus and method for the insertion of a medical tool, for example a needle, within the human body. The apparatus and method are particularly useful in prostate brachytherapy and in prostate biopsy. The apparatus comprises a telescoping guide universally coupled to a first and second positioning means that are used to automatically and/or manually position the guide at a desired needle insertion trajectory. Automatic positioning of the guide is accomplished with reference to three-dimensional transrectal ultrasound images that can also be used to show needle insertion in real-time. The apparatus may be manually positioned in the approximate insertion trajectory and then a computer interconnected with the apparatus may be used to achieve the final trajectory based upon the ultrasound images. The apparatus is particularly useful in cases where multiple needles are to be inserted into a small target area and in cases where pubic arch interference prevents direct access to the target area.

### FIELD OF THE INVENTION

The invention relates to the guiding of medical tools for insertion within the body of an animal. More particularly, the invention relates to an apparatus and method for positioning a guide for use in manually inserting a medical tool, such as a needle, within a human body at a particular angle in three-dimensional space, usually determined with reference to a medical image of an interior of the body. The guide may be automatically positioned by a computer and/or manually positioned. The apparatus and method are particularly useful as an aid in needle placement during the treatment of prostate cancer by brachytherapy or during prostate biopsy procedures.

### BACKGROUND

There are a number of medical procedures where it is desirable to insert a tool within a human body to a precise position based upon medical imagery. An example of such a procedure is prostate brachytherapy, where radioactive seeds are implanted within a cancerous prostate using a long needle that is inserted into the prostate from outside the body. Ultrasound images are used to determine the location and size of the prostate and a plate having a rectangular array of regularly spaced holes is used as a guide in inserting the needle at approximately the right location. An ultrasound transducer inserted within the rectum is sometimes used to provide real-time imagery as an aid in locating the prostate. This approach relies considerably upon the surgeon's skill in locating the prostate and is not particularly accurate in terms of seed placement.

Recent advances in ultrasound imaging equipment and techniques have allowed increasingly sophisticated models of the prostate to be produced. One such advancement is three-dimensional ultrasound imaging, which allows a three-dimensional model of the prostate to be assembled from a series of two-dimensional images. Accurate targeting of seed placement based upon these three-dimensional models can dramatically increase the efficacy of brachytherapy and therefore improve the survival of prostate cancer patients. However, limitations in the array-based needle guidance technique make it difficult if not impossible to accurately place the seeds in their target location. This is especially true in advanced prostate cancer cases, where enlargement of the prostate often causes a significant portion of the target area to be occluded by the pelvic arch.

It would be desirable to have an apparatus for accurately positioning a needle guide in three-dimensional space that allows the needle to be accurately inserted into the prostate at an angle that clears the pelvic arch and allows seeds to be precisely placed in their target location. To further improve the accuracy of seed placement, it would be desirable to have the guide positioning apparatus interfaced with a computer that automatically adjusts the position of the guide to the optimal location for placing each seed based upon analysis of an ultrasound image. Real-time feedback of ultrasound imagery to the computer would also be desirable in that it would allow the computer to make micro adjustments to the needle guide position in order to accurately place the seeds. Since many patients and surgeon's are wary of robotic surgical procedures, it is desirable that the needle guide simply position the needle for manual insertion by the surgeon and that the apparatus has sufficient override functionality to allow the surgeon to manually adjust the guide position without damaging the apparatus or endangering the patient.

U.S. Pat. No. 6,659,956, entitled “Medical Instrument Positioner”, describes a stage for a medical instrument that could include an ultrasound probe. The patent describes a method for translating a medical device along the axis of the stage. It does not describe the placement or use of a needle or other invasive medical device. U.S. Pat. No. 5,931,786, entitled “Ultrasound Probe Support and Stepping Device”, is similar to U.S. Pat. No. 6,659,956, but also includes rotation of an ultrasound probe in addition to translation. This device also has a template mount for needle insertion. No means is disclosed for guiding a needle to an intended target within the body.

U.S. Pat. No. 6,544,176, entitled “Computer Guided Cryosurgery”, describes a cryosurgery procedure involving a method for acquiring medical images, use of a computer system to plan appropriate placement of cryoprobes, placement of the probes using a template, and image-feedback on the appropriateness of the placement. This patent makes no reference to a means for guiding placement of medical tools and relies solely on a static template.

U.S. Pat. No. 5,494,039, entitled “Biopsy Needle Guide and Method for Use in Prostate Cryosurgery”, describes a cryosurgery placement technique wherein a needle guide is manually adjusted to a desired position, a needle is inserted, and the cryoprobe is inserted thereafter. This device can only be used manually. The angle of the needle is fixed relative to the probe axis, and thus cannot be adjusted in three-dimensional space to reach an occluded target within the human body.

U.S. Pat. No. 4,899,756, entitled “Articulating Needle Guide for Ultrasound Imaging and Method of Using the Same”, describes a fully manual needle guide for use with a surface ultrasound transducer. U.S. Pat. No. 6,695,786, entitled “Guide and Position Monitor for Invasive Medical Instrument”, expands on U.S. Pat. No. 4,899,756 by including position encoding devices. However, the devices disclosed in these documents are neither automatically operable, nor compatible with trans-rectal ultrasound probes.

The publication, Gabor Fightinger; Everette C. Burdette; Attila Tanacs; Alexandru Patriciu; Dumitru Mazilu; Louis L. Whitcomb; Dan Stoianovici; “Robotically assisted prostate brachytherapy with transrectal ultrasound guidance—Phantom experiments”, Brachytherapy 5 (2006) pp. 14-26, describes two robots for assisting in trans-rectal ultrasound guided prostate brachytherapy. The first robot described has inordinate flexibility of movement; the robot's operating envelope can extend into the patient, creating significant risk, and requiring a complex control system with complicated redundancy. This robot requires significant calibration, since the motive part of the robot is decoupled from the imaging device. It also consists of only a single positioning means, which limits the degrees of freedom that are selectively available in choosing a desired insertion angle. The second robot can position a needle, but has no mechanism to allow release of the needle once it is inserted. Furthermore, the structural supports of the robot's stage mechanism inhibit the inclusion of any kind of needle release mechanism. Since most brachytherapy procedures require the insertion of multiple needles, this makes the robot essentially un-useable in a clinic setting. This robot cannot be manually controlled, and the design also prohibits sterilization, a necessity for surgery or invasive therapy.

Prior art needle guides have suffered from limited positional accuracy, do not allow needle release for multiple needle placement and/or are either only manually adjustable or only robotically adjustable. The need therefore exists for an improved apparatus and method for guiding insertion of a medical tool, preferably in a manner permitting both manual and automatic position adjustment.

### SUMMARY OF THE INVENTION

According to an aspect of the present invention, there is provided an apparatus for the three-dimensional positioning of a guide for manual insertion of a medical tool within a body, the apparatus comprising: an alignment axis; a guide axis aligned with the guide; a first plane orthogonal to the alignment axis; a second plane orthogonal to the alignment axis and parallel to the first plane, the second plane spaced apart from the first plane along the alignment axis; a first positioning means for positioning a first guide point on the first plane, the guide axis passing through the first guide point; a second positioning means for positioning a second guide point on the second plane, the guide axis passing through the second guide point; and, the first and second positioning means separately adjustable in order to provide a pre-determined angular relationship between the guide axis and the alignment axis.

According to another aspect of the present invention, there is provided an apparatus for the three-dimensional positioning of a guide for manual insertion of a medical tool within a body, the apparatus comprising: at least one positioning means attached to the guide; at least one motor connected to each positioning means, the motor comprising: an armature; a gear shaft connected with the armature through a set of enmeshed gears; a manual adjustment knob connected with the gear shaft; a slip clutch connecting the gear shaft and the positioning means; and, the manual adjustment knob rotatable to manually adjust the positioning means while the motor is in operation without overdriving the motor.

According to yet another aspect of the present invention, there is provided a method of positioning a guide for manual insertion of a medical tool within a body, the method comprising: obtaining a medical image of an interior of the body; providing the medical image to a computer in a digital form; determining with the computer a desired angle for manual insertion of the tool based upon analysis of the image; and, automatically positioning the guide at the desired angle using instructions provided by the computer to a motorized guide positioning apparatus.

The first and/or second positioning means may be manually, automatically, or both manually and automatically adjustable. The first and/or second positioning means may comprise a means of adjusting the distance between the first guide point and the alignment axis and a means of adjusting the polar position of the first guide point on the first plane by rotating a first gear about a first rotation axis parallel to or collinear with the alignment axis. The means of adjusting the distance between the first guide point and the alignment axis may comprise: a first link member having a first end pivotally attached to a first crank member of the first gear and having a second end; a second gear spaced apart from the first gear along the alignment axis and rotatable about a second rotation axis parallel to or collinear with the alignment axis; a second link member having a first end pivotally attached to a second crank member of the second gear and having a second end pivotally attached to the first link member between its first and second ends at a link pivot point; and, the first and second gears rotatable to adjust the distance between the first guide point and the alignment axis. In one embodiment, the first link member and the second crank member are parallel and the first crank member and second link member are parallel, thereby forming a parallelogram linkage, with the first and second rotation axes being collinear. The second end of the first link member may comprise a universal joint or a spherical joint for connecting the guide means with the first link member.

The first and/or second positioning means may comprise a first locking means to selectively prevent movement of the second link member relative to the first link member about the link pivot point. The first and/or second positioning means may comprise a second locking means to selectively prevent movement of at least the first or second gear about its respective rotation axis. The guide may telescope between the first and second guide points and the guide may be lockable to selectively prevent telescoping. The first and second locking means and the lockable guide may co-operate to prevent movement of the first and/or second positioning means once a desired angle of tool insertion has been established. The guide may further include release means operable to release the tool from the guide without adjusting the position of the tool or the apparatus. This may be used, for example, to allow the tool to be removed from the apparatus without adjusting the position of the guide.

The angular relationship between the guide axis and the alignment means may be determined with reference to a medical image of an interior of the body. The medical image may comprise an ultrasound image and the apparatus may include means to facilitate the taking of real-time ultrasound images, for example an ultrasound transducer for insertion into the body along the axis of alignment. The ultrasound image may comprise a three-dimensional ultrasound image. The apparatus may further comprise one or more motors connected to the first and/or second positioning means and a computer interconnection means. The computer interconnection means may comprise a digital data connection or an analog connection for providing signals to the motor(s) for use in automatically adjusting the position of the guide. The position of the guide may be adjusted based upon feedback provided by real-time ultrasound images and/or other medical images.

The armature of the motor may be eccentric relative to the gear shaft and the set of enmeshed gears may comprise at least two eccentrically mounted gears. The apparatus may comprise a position encoder for determining the rotational position of the gear shaft and this rotational position may be fed back to the computer for use in determining any adjustments required in attaining the desired angle of insertion. The motor is designed such that the apparatus may be manually adjusted while the motor is in operation without backdriving or overdriving the motor. This advantageously permits the apparatus to be coarsely adjusted by a surgeon into the approximate position to facilitate proper alignment and/or allows a fine adjustment to be made by the surgeon using the manual adjustment knob, thereby giving the surgeon more control over the procedure. The manual adjustment feature also provides a safety override in the event of failure of the apparatus.

### BRIEF DESCRIPTION OF THE DRAWINGS

Having summarized the invention, preferred embodiments thereof will now be described with reference to the accompanying drawings, in which:

FIG. 1a is an illustration of a prior art needle insertion apparatus;

FIG. 1b is another illustration of the prior art needle insertion apparatus of FIG. 1 a;

FIG. 2a is an ultrasound image showing needle insertion in saggital view during a prostate brachytherapy procedure;

FIG. 2b is an ultrasound image showing needle insertion in coronal view during a prostate brachytherapy procedure;

FIG. 2c is an ultrasound image showing needle insertion in transverse view with the needle projected during a prostate brachytherapy procedure;

FIG. 3a is a front perspective view of an embodiment of the apparatus of the present invention;

FIG. 3b is a rear perspective view of the embodiment of FIG. 3a;

FIG. 4a is a partial rear perspective view of the embodiment of FIG. 3a in a first position;

FIG. 4b is a partial rear perspective view of the embodiment of FIG. 3a in a second position;

FIG. 5a is a partial rear perspective view of the embodiment of FIG. 3a, overlaid with a schematic illustration of the pivot points in the positioning means;

FIG. 5b is a kinematic diagram of the embodiment of FIG. 3a, illustrating the reference geometry;

FIG. 6a is an exploded rear perspective view of the first positioning means of the embodiment of FIG. 3a;

FIG. 6b is a kinematics diagram corresponding to FIG. 6a;

FIG. 6c is another kinematics diagram corresponding to FIG. 6a;

FIG. 7 is an exploded rear perspective view of a portion of the first positioning means of the embodiment of FIG. 3a;

FIG. 8a is an exploded rear perspective view of the first positioning means of the embodiment of FIG. 3a, illustrating the locking means;

FIG. 8b is an exploded rear perspective view of the telescoping guide of the embodiment of FIG. 3a, further illustrating the locking means;

FIG. 9 is an exploded perspective view of an embodiment of a motor for use in the present invention;

FIG. 10 is an exploded perspective view of an embodiment of a needle release mechanism for use in the present invention;

FIG. 11 is an exploded perspective view of another embodiment of a needle release mechanism for use in the present invention;

FIG. 12 is a flowchart illustrating a method of use of the apparatus according to the present invention;

FIG. 13 is a continuation of the flowchart of FIG. 12; and,

FIG. 14 is a further continuation of the flowchart of FIG. 12.

### DETAILED DESCRIPTION

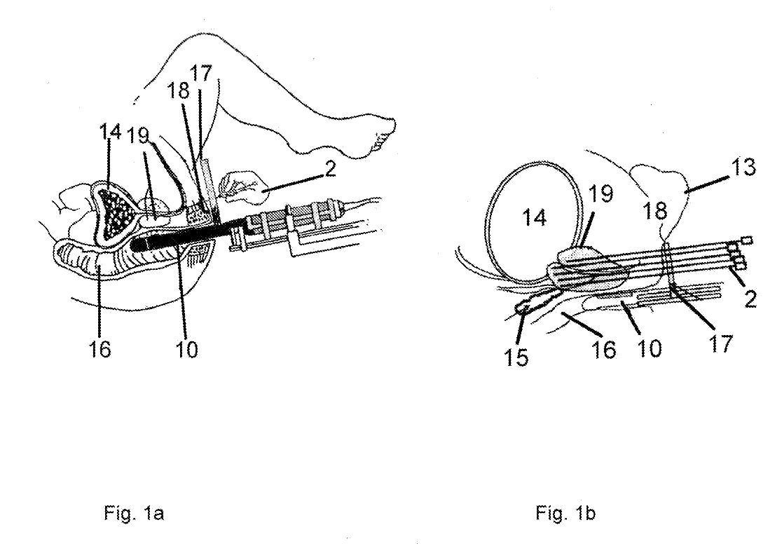

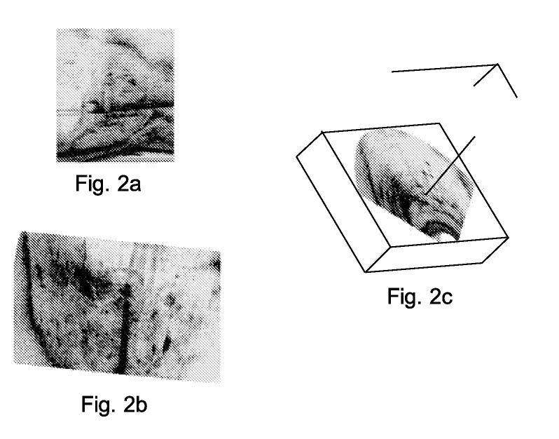

Referring to FIGS. 1a and 1b, during prostate brachytherapy a needle 2 used for the implantation of radioactive seeds is inserted into the prostate 19 through the perineum 18 of a supine patient. Also shown are the rectum 16, seminal vesicle 15, bladder 14, and scrotum 13. An ultrasound transducer 10 is inserted trans-rectally to provide real-time images of the needle 2 during insertion. Preferably, the ultrasound transducer 10 comprises a side firing linear array transducer coupled to a rotational mover for creating three-dimensional (3-D) trans-rectal ultrasound (TRUS) images. In creating the 3-D TRUS images, the mover rotates the transducer about its longitudinal axis in 1° increments through a 100° arc to generate a fan-shaped scan. As the transducer is rotated, two-dimensional (2-D) images are digitized by a frame grabber and stored in computer memory for reconstruction into a 3-D image immediately following acquisition. Exemplary images are provided in FIGS. 2a, 2b and 2c. The generation of 3-D ultrasound images is further described in U.S. Pat. Nos. 5,842,473 and 5,964,707, which are hereby incorporated by reference.

A needle template 17 comprising a rectangular array of uniformly spaced apart holes is provided in a spatially fixed relationship to the ultrasound transducer 10. By selecting an appropriate hole in the template 17, one or more needles 2 may be inserted into the prostate 19 using the ultrasound imagery as a guide. This prior art method relies to a certain extent upon trial and error to achieve proper needle placement. Since it is often necessary to insert more than one needle, the angle of needle insertion must be selected to prevent interference between successive needles; the needle template 17 is inherently limited in the angles that can be attempted and it is difficult to achieve proper seed placement at extreme angles. Furthermore, in many cases the pubic arch occludes a portion of the prostate and relatively extreme angles are needed to place seeds behind the arch. For these reasons and others, the prior art needle placement method using a fixed template 17 has certain surgical limitations.

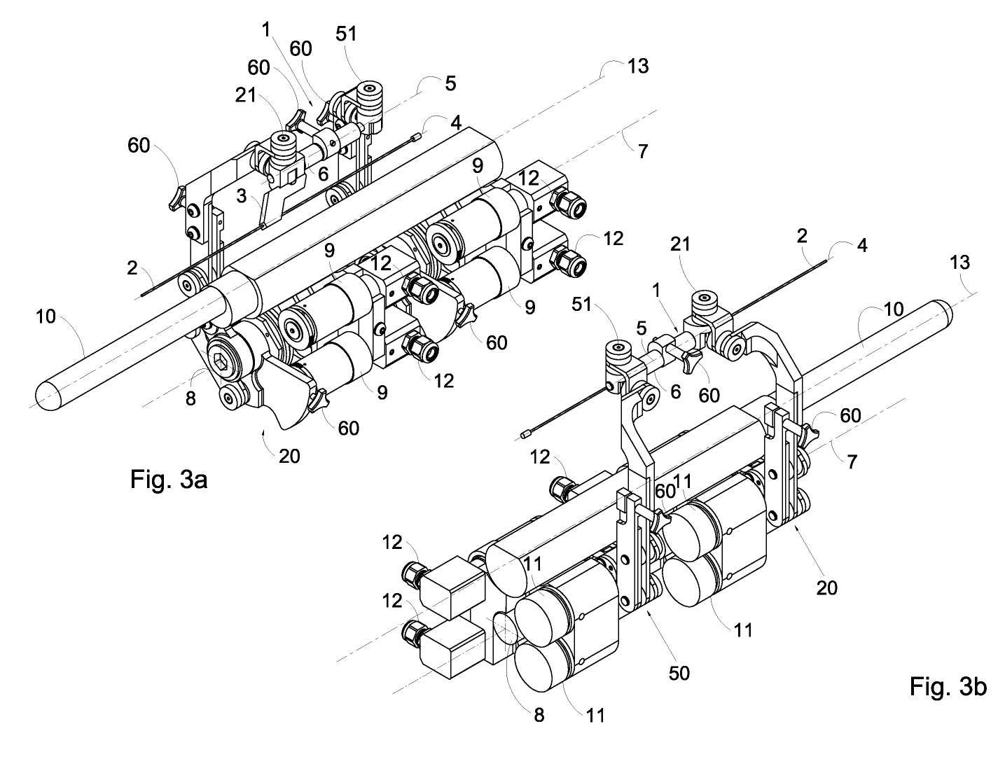

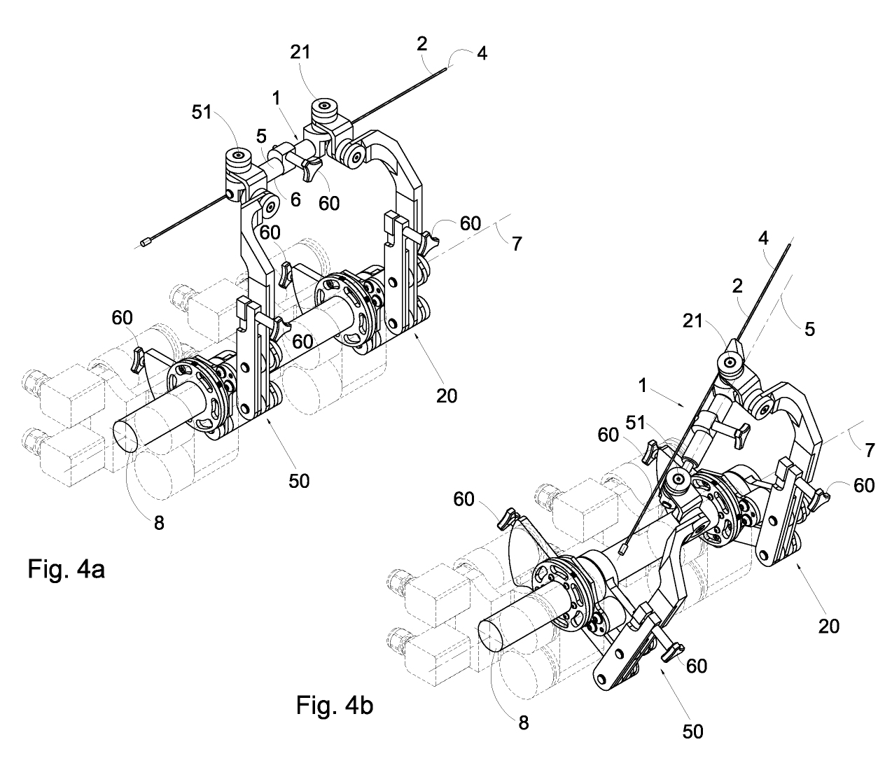

Referring to FIGS. 3a, 3b, 4a and 4b, a positionable guide apparatus according to the present invention comprises a telescoping guide 1 for insertion of a tool into a body (not shown). In the embodiment shown, the tool is a needle 2 for the implantation of radioactive seeds during prostate brachytherapy. A needle holder 3 having an aperture complementary to the needle diameter depends from the guide 1. A needle axis 4 passes through the aperture and is parallel to and offset from a guide axis 5 passing through the telescoping portion 6 of the guide 1. The guide 1 is suspended from a first positioning mechanism 20 and a second positioning mechanism 50 by means of a pair of hook joints 21 and 51. When the first and second positioning means 20, 50 are differentially moved, the hook joints (also known as universal joints) allow the guide 1 and, by definition, the guide axis 5 to be adjusted to any desired angular orientation in three dimensional space relative to an alignment axis 7 aligned with a central shaft 8. Since the needle axis 4 is parallel with the guide axis 5, it is accordingly also adjusted to the desired angle. A plurality of motors 9 are mounted on the central shaft 8 and are provided to facilitate automatic adjustment of the first and second positioning means 20, 50 under instruction of a computer (not shown) electrically connected with the apparatus. A position encoder 11 is mounted to the shaft 8 and provided in association with each motor 9 to feed back the rotational position of the motor to the computer. The central shaft 8 provides a fixed reference for all movements of the apparatus and can be used to secure the apparatus to a surgical table or other operating room fixture.

A transrectal ultrasound transducer 10 may optionally and preferably be provided for use in taking real-time images of the prostate during insertion of the needle 2. The transrectal transducer 10 is preferably mounted to the central shaft 8 with an insertion axis 13 that is parallel to or coaxial with the alignment axis 7 to simplify calculation of the desired angle of tool insertion with reference to the centerline of the images being taken. The images are preferably fed back to the computer or computer system that is being used to automatically control the position of the guide means so that any changes required to the angle of insertion based upon the images may be readily implemented.

It should be noted that the entire apparatus is designed to be sterilizable in compliance with applicable medical device regulations; accordingly, all electrical connections are provided via conduits attached to the apparatus through fluid tight fittings 12 selected for compatibility with a variety of commonly known sterilization fluids and conditions.

At least one locking knob 60 is provided on each of the guide 1, the first positioning means 20 and the second positioning means 50. These locking knobs 60 may be rotated in order to selectively prevent their associated mechanism from moving after a desired tool insertion angle has been set. Choosing a desired combination of locking knobs 60 allows the surgeon a number of degrees of freedom in adjusting or selectively preventing the movement of the apparatus, for example to minimize the potential for inadvertent misalignment of the guide means after a desired angle has been set. The function of the locking knobs 60 in conjunction with their associated locking means will be further described hereinafter.

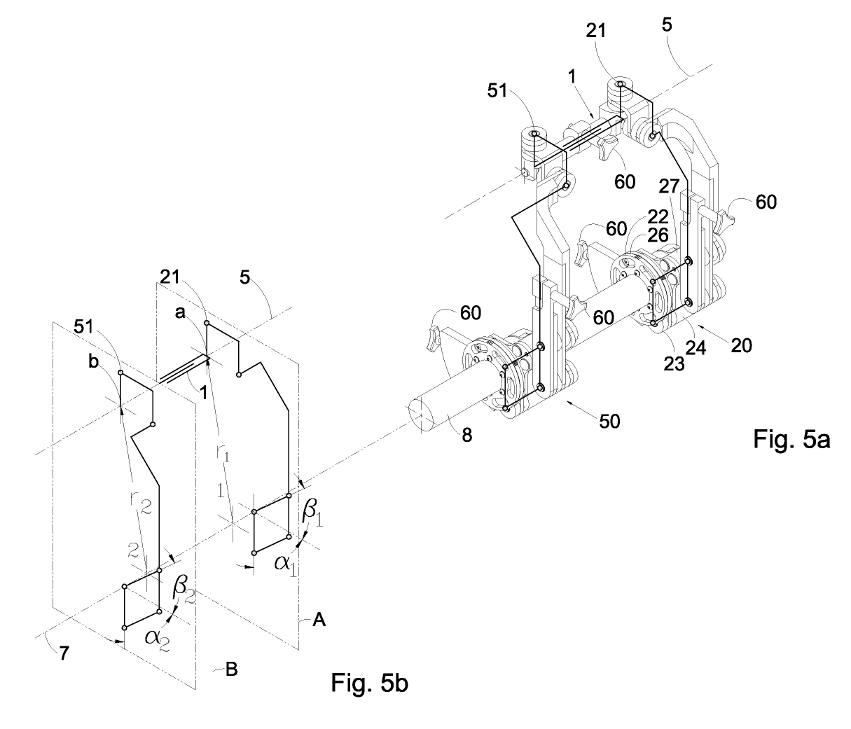

Referring to FIGS. 5a and 5b, a first plane A is provided orthogonal to the alignment axis 7 and a second plane B is provided orthogonal to the alignment axis 7 spaced apart from and parallel to the first plane. A first guide point, denoted as a, is located on the guide axis 5 at a position corresponding to the first positioning means 20 and a second guide point, denoted as b, is located on the guide axis at a position corresponding to the second positioning means 50. The first and second positioning means 20, 50 each comprise a means of adjusting the polar position of their respective guide points a, b on their respective planes A, B by rotation about the alignment axis 7, as well as a means of adjusting the distance between the guide points a, b and the alignment axis 7. In the embodiment shown, these two means are provided together in a parallelogram linkage, which will be further described hereinafter. The guide points a, b are located at the center of the hook joints 21, 51 and the guide 1 is permitted to telescope between the guide points. As the first and second positioning means 20, 50 are operated to adjust the location of the guide points a, b on their respective planes, the hook joints 21, 51 and telescoping guide 1 enable the guide axis 5 to adopt a corresponding angular orientation with reference to the alignment axis 7. A desired angle of tool insertion can therefore be attained by calculating the corresponding positions of the guide points a, b on the planes A, B in a polar co-ordinate system.

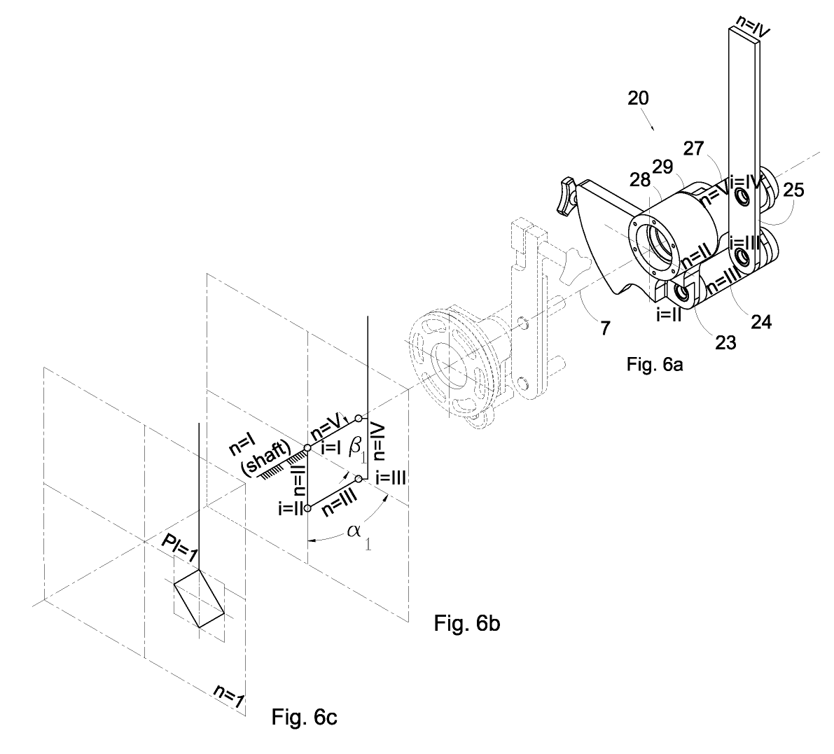

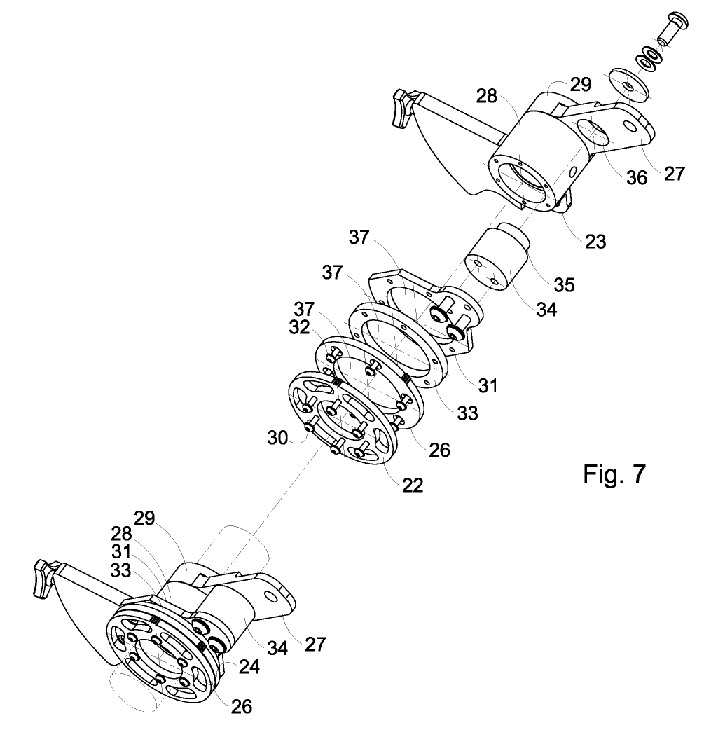

Referring additionally to FIGS. 6a, 6b and 7, the parallelogram linkage of the first positioning means 20 comprises a first gear 22 with a first hub 28 rotationally mounted to the central shaft 8 concentric with the alignment axis 7 attached thereto by means of fasteners 30. The center of rotation of the hub 28 about the alignment axis 7 is denoted schematically as i=1. The first hub 28 includes a first crank member 23 extending therefrom and having a first end of a first link 24 pivotally attached thereto at a point radially spaced apart from the alignment axis 7; this point is denoted schematically as i=2. A second end of the first link 24 is pivotally attached to a first end of a second link 25 at a point denoted schematically as i=3.

A second gear 26 is attached to a stand-off bushing 31 by means of fasteners 32. A spacer 33 is sandwiched between the second gear 26 and the stand-off bushing 31. The second gear 26, spacer 33 and stand-off bushing 31 all have concentrically aligned interior apertures 37 of sufficient diameter to fit over a posterior portion of the first hub 28 and are permitted to rotate relative to the first hub 28. A posterior end of a stand-off 34 is attached to the stand-off bushing 31 such that the stand-off 34 is spaced apart from the shaft 8 and parallel thereto. The stand-off 34 comprises an anterior end 35 having a reduced diameter that is fitted within a complementary aperture 36 of a second crank member 27. The second crank member 27 extends outwardly from a second hub 29 that is rotationally mounted to the central shaft 8 concentric with the alignment axis 7. Rotation of the second gear 26 about the posterior portion of the first hub 28 causes the stand-off bushing 31 to co-rotate, which in turn causes the stand-off 34 to orbit about the shaft 8 and act upon the second crank member 27, thereby inducing co-rotation of the hub 29 about the alignment axis 7.

The second crank member 27 is pivotally attached to the second link 25 at a link pivot point, denoted schematically as i=4, that is radially spaced apart from the alignment axis 7 and located on the second link 25 between its first end and a second end. The second end of the second link 25 extends in a generally upward direction and is connected either directly or indirectly to the hook joint 21. In the embodiment shown, a parallelogram linkage is formed by two sets of parallel members; the first link 24 and the second crank member 27 are parallel, along with the first crank member 23 and the second link 25. It will be understood by persons skilled in the art that these members need not necessarily be parallel to achieve the function of a parallelogram linkage.

Returning briefly to FIG. 5b, the angle α₁ is the rotational angle of the first gear 22 defined between the first crank member 23 and a fixed reference perpendicular to the alignment axis 7, whereas the angle β₁ is the rotational angle of the second gear 26 defined between the second crank member 27 and the same fixed reference. The distance between the first guide point a and the alignment axis 7 is defined by r₁ and the angle between r₁ and the fixed reference is depicted as θ₁. Rotation of the first and second gears 22, 26 causes adjustment of the angles α₁ and β₁, resulting in “opening” or “closing” of the parallelogram linkage, and may be used to adjust the position of the first guide point a on the first plane A. Depending on the degree of counter or co-rotation of the first and second gears, this change in position may relate to the rotational position of the guide point a (a change in the angle θ₁), the distance between the guide point a and the axis of alignment (a change in r₁), or both simultaneously. For example, co-rotation of the first and second gears 22, 26 by the same amount causes the sum of the angles α₁ and β₁ to remain constant; this results in a change in θ₁ without adjusting r₁. The second positioning means 50 functions in a similar manner to the first positioning means 20.

Referring now to FIGS. 6b and 6c, the simplified kinematics' model illustrated in FIG. 6b shows a symbolic representation of the linkage and the nature of its interconnections, which can be simplified further by reducing all of the components into higher-order subassemblies. Applying the Kutzbach criterion:

$$l ( n - 1 ) + {\sum }_{i = 1}^{j} ( {p}_{i} - 1 ) ( {f}_{i} - l )$$

to the pinned parallelogram schematic reveals that this linkage can be reduced to a non-parametric planar joint of mobility 2, where n is the total number of elements connected, ƒᵢ is the mobility of the joint and l is the mobility one link can have relative to the other. Since this is a planar linkage, each component can have a total of 3-degrees of freedom.

Reconstructing the kinematics' chain from the spatial components derived in the previous expression generates the simplified closed chain in FIG. 6c, where the planar joint P_{L} (Pl) has replaced the pinned parallelogram. Applying the Kutzbach criterion to the spatial linkage gives a total system mobility of 4. In this case, the variable l in the spatial system has a total of 6 degrees of freedom.

The non-parametric model in FIGS. 6b and 6c reveals that the mobility of the system is unaffected by the type of joint represented by P_{L} (Pl). This element can represent a spherical linkage, or any other type of assembly with mobility 2, to produce a variation on the parallelogram linkage. The length of each link can be dimensioned to suit an application where the operating envelope needs to be altered. The links and/or cranks need not necessarily be straight, but could be curved, bent, or any other shape.

All of the apparatus' functions are mechanically coordinated in such a manner where the mobility can be systematically reduced to zero degrees of freedom. Once the needle guide is in the desired position, the degree of freedom can be reduced to zero, thus making the device rigid. At this stage, the needle can be confidently inserted without concern that the apparatus will move. Five mechanical brakes are integrated into the design of the system to constrain each of its decoupled movements, allowing this progressive reduction in degrees of freedom. These brakes include a concentric ring brake and a parallel brake on each of the first and second positioning means 20, 50 and a guide brake on the telescoping guide 1, all of which will be further described hereinafter.

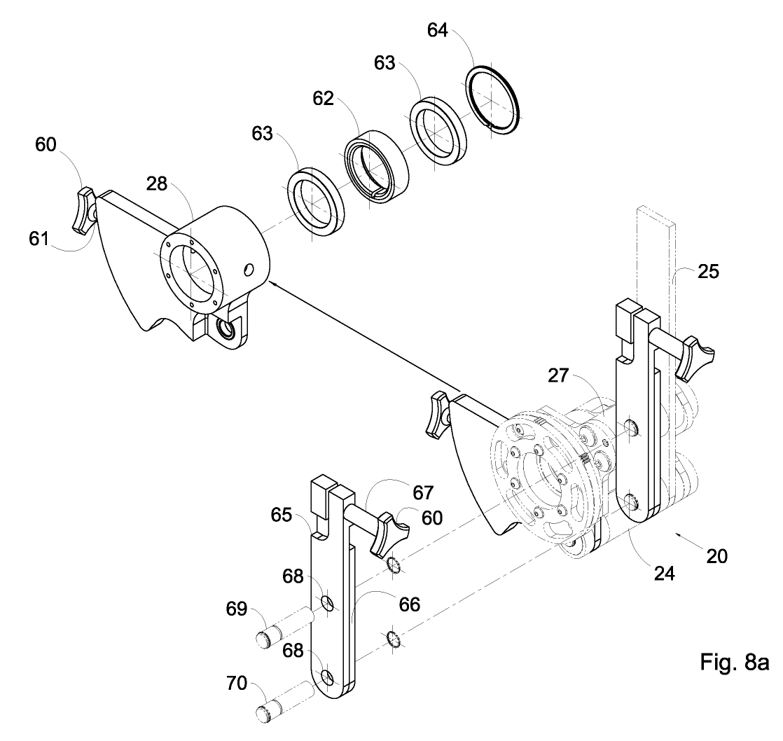

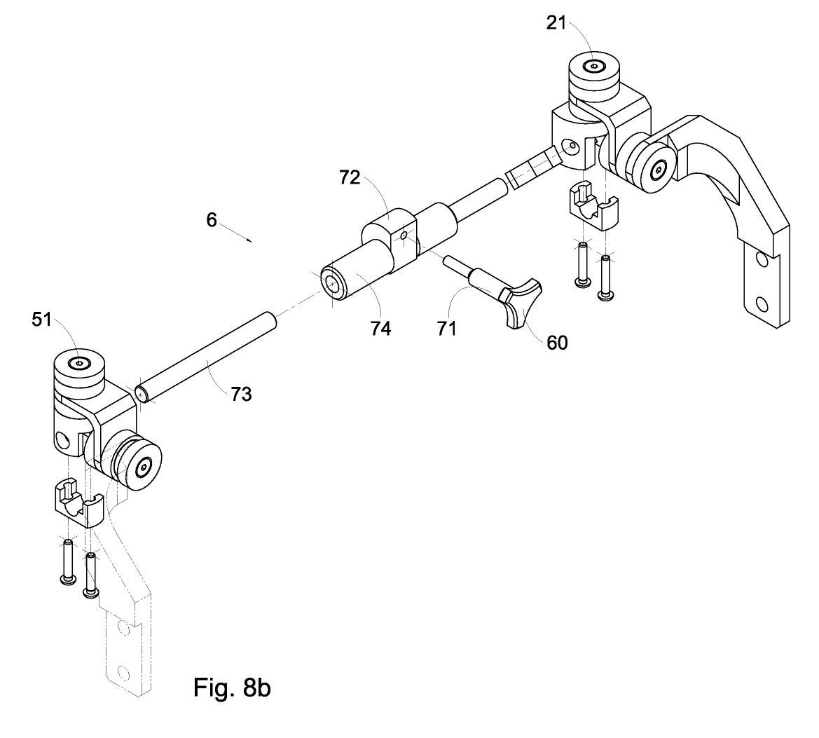

Referring to FIG. 8a, the front positioning means 20 includes a concentric ring brake integrated with the first hub 28. The concentric ring brake comprises a locking knob 60 attached to a clamping screw 61 that exerts pressure upon a split ring 62 that resides inside the first hub 28 between bearings 63. A snap ring 64 located within a recessed groove (not shown) retains the bearings 63 and split ring 62 within the first hub 28. Rotating the locking knob 60 to tighten the clamping screw 61 causes the split ring 62 to collapse upon the base shaft 8. The frictional force generated by this clamping action prevents rotation of the first hub 28 (and hence, the first gear 22) about the alignment axis 7; the angle α₁ therefore remains constant. However, the second gear 26 and its associated hub 29 are still permitted to rotate and the angle β₁ can still be adjusted. Application of the concentric ring brake therefore constrains the first positioning means 20 to function as a four-bar linkage. This constraint applies equally to manual or automatic adjustment and the motor 9 associated with the first gear 22 is prevented from operating while the motor associated with the second gear 26 is able to function in the normal manner.

The front positioning means 20 further includes a parallel brake comprising a first brake plate 65, a second brake plate 66, a locking knob 60 and a scissor screw 67. Each brake plate 65, 66 includes a pair of spaced apart apertures 68 for receiving first and second linkage pivot pins 69, 70. The first linkage pivot pin 69 is used to connect the second crank member 27 to the second link 25 at i=4 and the second linkage pivot pin 70 is used to connect the first link 24 to the second link 25 at i=3. The first linkage pivot pin 69 is secured to the second crank member 27 and the second linkage pivot pin 70 is secured to the first link 24, while the second link 25 is permitted to pivot about the linkage pins 69, 70 during movement of the first positioning means 20. When the parallel brake is installed, the apertures 68 of the two plates are aligned and the brake plates 65, 66 abut one another. Rotating the locking knob 60 to tighten the scissor screw 67 urges the plates to rotate in opposite directions about the linkage pivot pin 70, creating a shearing action against the linkage pivot pin 69 that prevents it from rotating; this in turn constrains the parallelogram linkage such that the relative positions of the link and crank members is fixed and the sum of the angles α₁ and β₁ remains constant. When the parallel brake is engaged, the parallelogram linkage is constrained such that the first and second gears 22, 26 are only able to rotate in unison (at the same speed and in the same direction) about the alignment axis 7; consequently, only the angle θ₁ is permitted to change, without variation of r₁. This constraint applies equally to manual or automatic adjustment of the apparatus. When both the parallel brake and the concentric ring brake are applied, the first positioning means 20 is locked and the position of the first guide point a on the first plane A is fixed.

When the first positioning means 20 is locked, the second positioning means 50 may be able to move freely, partially constrained or fully constrained. Re-positioning of the second guide point b on the second plane B allows the trajectory of the needle 2 to be adjusted and is useful in maneuvering the needle around obstacles observed on the ultrasound image. Many surgeons prefer to conduct these trajectory adjustments manually, but they could also be completed automatically through operation of the motors 9 associated with the second positioning means 50 under instruction of the computer associated with the apparatus. Once the desired trajectory has been established, both the first and second positioning means 20, 50 are normally locked to fix the trajectory and permit insertion of the needle 2 along the desired path without inadvertently misaligning the apparatus. Locking of the first and second positioning means 20, 50 is also useful in the event of a failure in one or more of the motors 9 to ensure that no adverse outcomes result from any re-alignment of needle trajectory.

Referring to FIG. 8b, the telescoping guide brake comprises a locking knob 60 connected to a guide screw 71. Rotation of the locking knob 60 causes the end of the guide screw 71 to tighten a concentric ring claim 72 that frictionally engages the circumference of an inner portion 73 of the telescoping guide 6 that is located within a complementary outer portion 74. This restricts relative movement between the inner portion 73 and the outer portion 74 and prevents telescoping of the guide 1.

Application of the telescoping guide brake prevents relative movement of the guide points a, b on their respective planes and constrains the first and second positioning means 20, 50 to move in unison. This is particularly useful when both of the parallel brakes are engaged, as the first and second positioning means 20, 50 are then constrained to rotate in unison about the alignment axis 7. Once a desired trajectory has been established, application of the parallel brakes and guide brake allows the apparatus to be pivoted out of position, for example to replace a given needle with another type of needle or to continue with the procedure in the event of an electrical failure, without affecting the pre-established trajectory. Application of the telescoping brake by itself is also useful in regulating the speed of all of the motors 9 during the final stages of establishing a desired trajectory.

In manually adjusting the apparatus, the potential exists to move the first and/or second positioning means 20, 50 out of synchronization with the rotational position of the motor(s) 9 and to damage the motor(s) through overdriving or backdriving. These problems are addressed in the present invention through certain design features that allow the apparatus to be both manually and automatically adjusted, even while the motor(s) are in operation. The ability to manually adjust the apparatus is important from a surgical point of view, in that it allows the surgeon to intervene either to complete the procedure in the event of an electrical failure of the apparatus and permits the surgeon to make both coarse and fine adjustments during a procedure. Coarse adjustments are useful in moving the apparatus to the approximate desired position, with final positioning being determined either by the computer or manually by the surgeon with reference to the ultrasound image using the fine adjustment knobs.

To enable the computer to synchronize the rotational position of a given motor 9 with the actual position of the first or second positioning means 20, 50, the apparatus employs a separate position encoder 11 in association with that motor's respective gear. For example, manual adjustment of the first positioning means 20 causes the first and/or second gears 22, 26 to rotate; the amount of rotation is tracked by the position encoder 11 associated with that gear and is fed back to the computer. Since the motors 9 only operate if instructed to do so by the computer, the rotational position of the motor associated with a particular gear is also continuously tracked with reference to an initial physical calibration. By comparing the rotational position of a particular gear with the rotational position of its respective motor, the computer is able to synchronize the motor with the actual position of the positioning means so that further automatic adjustments to the location of the guide point on its respective plane produce the desired result. To further ensure positional accuracy, the motors 9 may each be equipped with another internal position encoder that feeds back the rotational position of the motor to the computer. This redundant motor position feedback helps ensure synchronization of the motor with the positioning means, even in the event of a software glitch or temporary power interruption to the computer system.

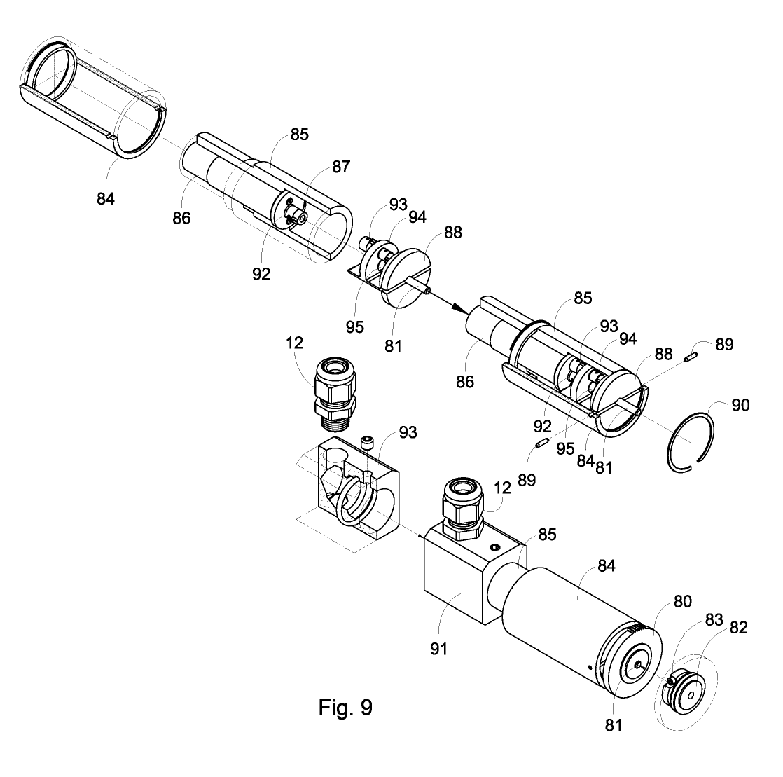

Referring to FIG. 9, each motor 9 comprises a drive gear 80 for engagement with a complementary gear of the first or second positioning means, for example, the first or second gears 22, 26. The drive gear 80 is connected with a drive shaft 81 of the motor via an adjustable slip clutch mechanism 82. Tightening the adjustment nut 83 of the slip clutch mechanism 82 increases the concentric clamping friction between the slip clutch mechanism and the drive shaft 81. Normally, the drive shaft 81 and drive gear 80 are coupled through the slip clutch 82 and rotate together in unison; however, forces applied to either the drive shaft 81 or the drive gear 80 that exceed the pre-set clamping friction cause decoupling, resulting in independent rotation of the two. Excessive forces may arise through interference between the positioning means and a physical obstacle (for example, another piece of equipment or a body part) or through aggressive manual re-positioning of the apparatus. The slip clutch is a safety mechanism that prevents damage to the apparatus, particularly as a first line of defence in preventing backdriving or overdriving of the motor 9, and reduces the likelihood of injury to patients or people operating the apparatus.

Each motor further comprises a drive housing 85 containing a sealed motor unit 86 having an armature 87 extending outwardly therefrom into an interior of the drive housing. A gear drive insert 88 is also inserted within the drive housing 85 and supports an unbalanced differential drive train which will be further described hereinafter. A fine adjustment knob 84 is placed over the drive housing 85 and the gear drive insert 88 is keyed thereto by means of pins 89. A snap ring 90 resides within a circumferential interior groove of the fine adjustment knob 84 and abuts the exterior face of the gear drive insert 88 to retain the fine adjustment knob in position. The drive shaft 81 extends outwardly from the gear drive insert 88 and is permitted to rotate relative thereto upon operation of the motor unit 86.

The motor unit 86 has a high resistance to externally induced rotation of the armature 87; in effect, the motor unit 86 is locked when not in operation, preventing backdriving during coarse manual adjustment. Similarly, the high resistance to external induced rotation prevents overdriving while the motor is in operation, permitting fine manual adjustment to take place concurrently with automatic adjustment if so desired. This resistance to rotation can be imparted either electrically, mechanically, or both. For example, the motor unit 86 may comprise a DC motor with a large internal magnetic coupling force that prevents undesired external rotation of the armature 87. In other embodiments, the motor unit 86 may comprise a stepping motor with similar features. The motor unit 86 may include a large internal gear ratio to mechanically amplify its resistance to rotation of the armature 87. In one embodiment, the internal gear ratio is from 300:1 to 900:1, preferably from 500:1 to 700:1, more preferably about 600:1. It is desirable the motor chosen for the motor unit 86 permits quantifiable rotation of the armature 87 so that an exact movement of the positioning means may be achieved under computer instruction. The motor unit 87 is sealed and resides within a sealed motor base 91 equipped with fluid tight connections 12 for conduits containing electrical power and/or positional signals. This permits the entire apparatus to be sterilized without damaging its electrical components.

Although the high resistance of the motor unit 86 to externally induced rotation is useful in preventing damage to the motor, it would also prevent rotation of the fine adjustment knob 84 if directly coupled to the armature 87. In order to permit manual adjustment to occur, the fine adjustment knob 84 is connected to the armature 87 through an unbalanced differential drive train. The unbalanced differential drive train comprises a set of at least four drive gears. The armature 87 is equipped with a first drive gear 92 enmeshed with a second drive gear 93. The second drive gear 93 is located on a common shaft with a third drive gear 94 that is enmeshed with a fourth drive gear 95 mounted on the drive shaft 81. The gear ratio of the differential drive train between the first drive gear 92 and the fourth drive gear 95 is preferably greater than one, more preferably greater than two, so that a rotation of the armature 87 translates into only a partial rotation of the drive shaft 81. For example, in one embodiment, the first drive gear 92 has twelve teeth, the second drive gear 93 has sixteen teeth (gear ratio 1:0.75=4:3), the third drive gear 94 has twelve teeth and the fourth drive gear 95 has sixteen teeth (gear ratio 1:0.75=4:3), producing an overall gear ratio of 16:9=1.78. In another embodiment, the first drive gear 92 has twelve teeth, the second drive gear 93 has twenty teeth (gear ratio 1:0.6=5:3), the third drive gear 94 has twelve teeth and the fourth drive gear 95 has twenty teeth (gear ratio 1:0.6=5:3), producing an overall gear ratio of 25:9=2.78.

During manual rotation of the fine adjustment knob 84, the gear drive insert 88 orbits about the armature 87 by virtue of its direct connection to the knob through pins 89. Since the motor unit 86 has a high resistance to externally induced rotation of the armature 87, the first drive gear 92 remains fixed in position. Accordingly, the second and third drive gears 93, 94 co-rotate in the same direction as the adjustment knob 84, while the fourth drive gear 95 counter-rotates. Although this has a tendency to cause the drive shaft 81 to rotate in the opposite direction to the rotation of the fine adjustment knob 84, due to the high gear ratio of the unbalanced differential drive train this counter-rotation is small compared with the orbital movement of the gear drive insert 88. The net effect is to cause the drive gear 80 to rotate in the same direction as the fine adjustment knob 84. This in turn permits manual movement of the positioning means, without backdriving or overdriving the motor unit 86, allowing manual adjustment to take place even while the motor unit is in operation.

The unbalanced nature of the drive train arises from the planetary relationship of the first drive gear 92 with the second drive gear 93 and the fourth drive gear 95 with the third drive gear 94. This planetary relationship tends to urge the gears out of enmeshment, creating a slightly non-parallel alignment between the axes of rotation of the gears. The wedging action produced by this non-parallel alignment causes a great deal of friction between the drive gears, even when not in operation. This friction is important in maintaining the positioning means in an upright orientation. Without this friction, the fine adjustment knob 84 could spin freely and the weight of the positioning means would backdrive the gear drive insert 88, causing the positioning means to collapse under its own weight. However, during manual fine adjustment, the friction of the unbalanced drive train is easily overcome and the surgeon is permitted to make the desired changes to the positioning means.

In performing certain procedures, in particular brachytherapy or needle biopsy procedures, it is often necessary to insert more than one tool (eg: needle, scope, etc.) into the body simultaneously. To facilitate these types of procedures, it would be desirable that the apparatus allows the tool to be released once inserted to the correct position in order that the apparatus is made free for use in inserting a subsequent tool. In this manner, a plurality of tools can be inserted to the desired position simultaneously during a procedure.

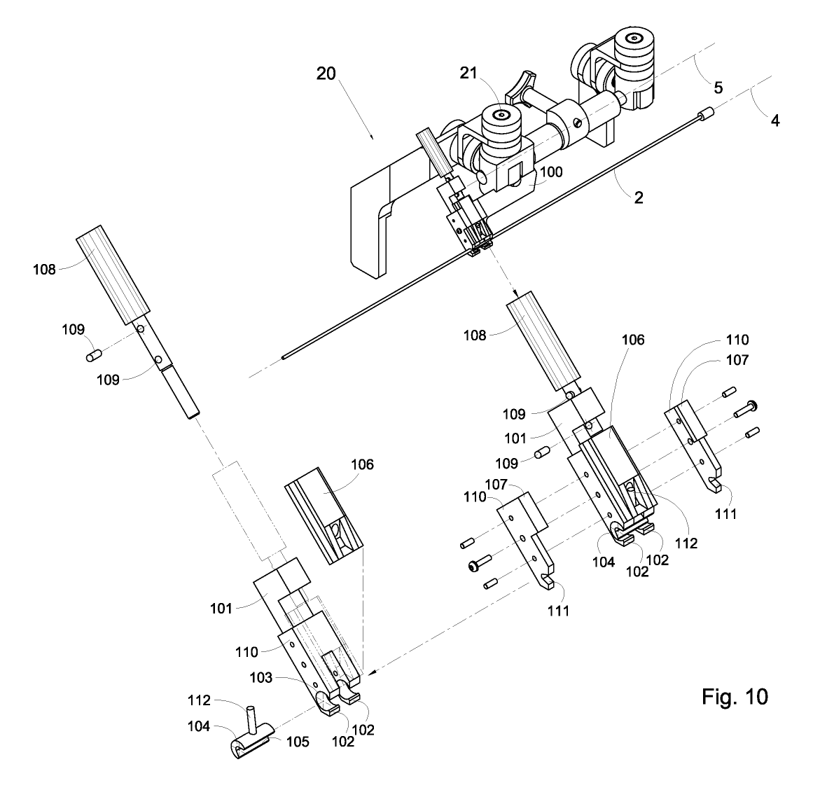

Referring to FIG. 10, a first embodiment of a tool release mechanism includes a stand-off 100 fixedly mounted to the first positioning means 20 proximal the hook joint 21 and extending towards the front of the apparatus. At the anterior end of the stand-off 100 is fixedly mounted a release block 101 having a pair of curved fingers 102 provided at one end thereof. The curved fingers 102 partially enclose an aperture 103 that is oversized compared with the tool (in this case, the needle 2) that is to be inserted therethrough. The non-enclosed portion of the aperture 103 provides a slotted chordal opening that is large enough to permit the needle 2 to be removed from the aperture in a direction perpendicular to the axis of insertion 4. A pair of side brackets 110 is secured to either side of the release block 101. Each side bracket 110 includes a crescent shaped slot 111 with an open end that is roughly aligned with the slotted chordal opening. When inserted within the aperture 103, the needle 2 seats at the apex of the crescent-shaped slots 111. Residing within the aperture 103 is a cylindrical clamping block 104 having an elliptical interior slot 105 with an upper end that is offset from the center of the aperture. Upon rotation of the clamping block 104, the upper end of the slot 105 approaches the center of the aperture 103 until it engages the needle 2 and wedges it against the apex of the crescent shaped slots 111. The clamping block 104 is counter-rotated slightly prior to performing a procedure in order to un-lock the needle 2 and permit longitudinal sliding of the needle 2 along the axis of insertion 4. The release mechanism is oriented such that, when the needle 2 is secured within the aperture 103, the axis of insertion 4 is parallel with the guide axis 5.

To facilitate rotation of the clamping block 104, a block crank 112 is located at the center of the block and extends perpendicularly to its axis of rotation. A slider block 106 is confined by slide plates 107 to slide along the release block 101 in response to screw mechanism 108. The screw mechanism 108 is equipped with stops 109 to prevent inadvertent over-tightening or disassembly of the release mechanism. Operation of the screw mechanism 108 causes the slider block to engage the crank 112 when slid along the release block 101, thereby rotating the clamping block 104 and securing the needle 2 within the aperture 103.

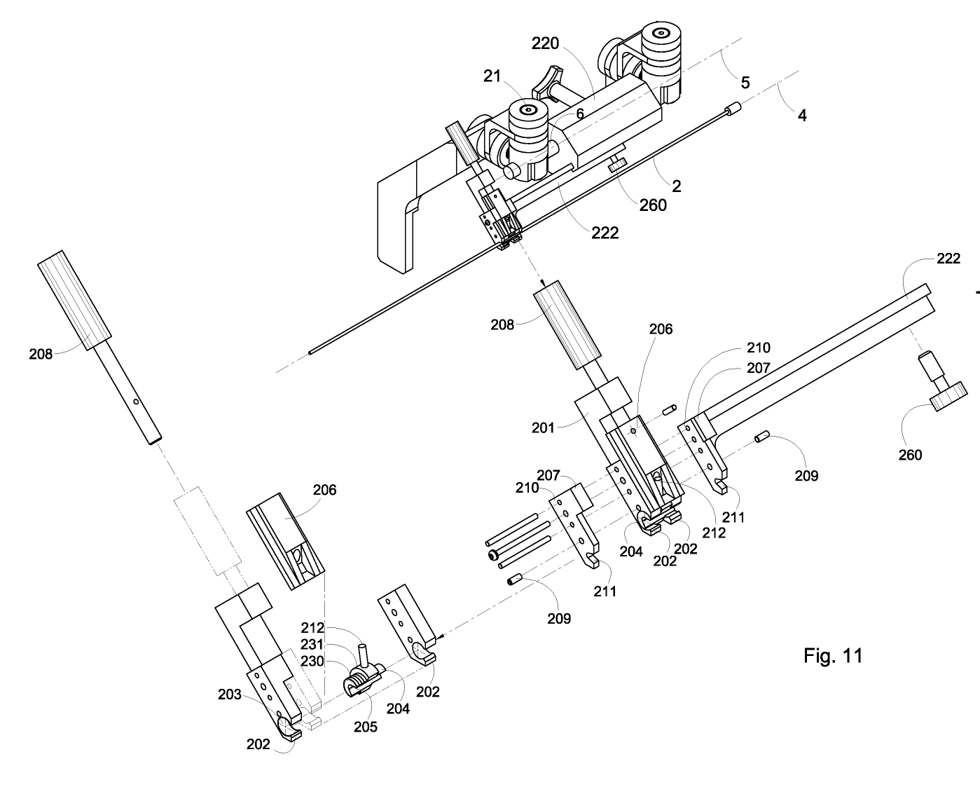

Referring to FIG. 11, in a second embodiment of a tool release mechanism a mounting block 220 is provided that permits the telescoping guide 6 to move therewithin along the guide axis 5. The mounting block 220 includes a T-shaped slot for receiving a complementary T-shaped mounting bar 222. The T-shaped mounting bar 222 is able to slide within the slot parallel to the guide axis 5. At the anterior end of the mounting bar 222 is fixedly mounted a release block 201 having a pair of curved fingers 202 provided at one end thereof. The curved fingers 202 partially enclose an aperture 203 that is oversized compared with the tool (in this case, the needle 2) that is to be inserted therethrough. The non-enclosed portion of the aperture 203 provides a slotted chordal opening that is large enough to permit the needle 2 to be removed from the aperture in a direction perpendicular to the axis of insertion 4. A pair of side brackets 210 is secured to either side of the release block 201. Each side bracket 210 includes a crescent shaped slot 211 with an open end that is roughly aligned with the slotted chordal opening. When inserted within the aperture 203, the needle 2 seats at the apex of the crescent-shaped slots 211. Residing within the aperture 203 is a cylindrical clamping block 204 having an elliptical interior slot 205 with an upper end that is offset from the center of the aperture. Upon rotation of the clamping block 204, the upper end of the slot 205 approaches the center of the aperture 203 until it engages the needle 2 and moves it toward the apex of the crescent shaped slots 211. The release mechanism is oriented such that, when the needle 2 is secured within the aperture 203, the axis of insertion 4 is parallel with the guide axis 5.

The clamping block 204 is equipped with a threaded end 230 that engages with complementary interior threads within the aperture 203 to move the block longitudinally along the axis of insertion 4 upon rotation. The clamping block 204 includes a central shoulder 231 that engages adjustable stops 209 as it is advanced or retracted along the axis of insertion 4. These stops 209 may be set to correspond to a particular diameter of needle 2 so that the clamping block 204 does not wedge the needle completely against the apex of the crescent-shaped slots 211. This obviates the need for loosening the clamping block 204 prior to performing a procedure.

To facilitate rotation of the clamping block 204, a block crank 212 is located at the center of the block on the shoulder 231 and extends perpendicularly to the axis of rotation. A slider block 206 is confined by slide plates 207 to slide along the release block 201 in response to longitudinal movement of a slide mechanism 208. Pushing or pulling the slide mechanism 208 causes it to act directly upon the block crank 212, thereby causing rotation of the clamping block 204. The slide mechanism 208 is permitted to operate between the two pre-set stop points 209 to control the degree of clamping achieved by the clamping block 204. The slide mechanism 208 may optionally include a bayonet style lock in place of or in addition to the fixed stops 209; this allows a plurality of clamping positions to be achieved in order to accommodate a plurality of different needle diameters and provides positive confirmation that the needle is secured within the aperture 203.

By loosening the locking knob 260, the second embodiment advantageously permits the entire release mechanism to be moved along the guide axis 5 so that it is either closer to or further away from the patient. Also, the second embodiment permits the entire release mechanism to be removed and replaced, for example in the event that a tool is needed with a larger diameter than can be accommodated by the aperture 203. The stops 209 are pre-set to a desired clamping condition, thereby obviating the need for releasing the needle by counter-rotation prior to performing the procedure. This advantageously increases the speed with which a needle may be inserted or removed from the mechanism as compared with the first embodiment, as the screw mechanism 108 is relatively time consuming to operate. However, the screw mechanism 108 has a wide range of adjustment and permits precise setting of the “feel” of the needle 2 as it slides through the aperture 103. The features of the first and second embodiments may be combined in various sub-combinations in order to attain desired advantages.

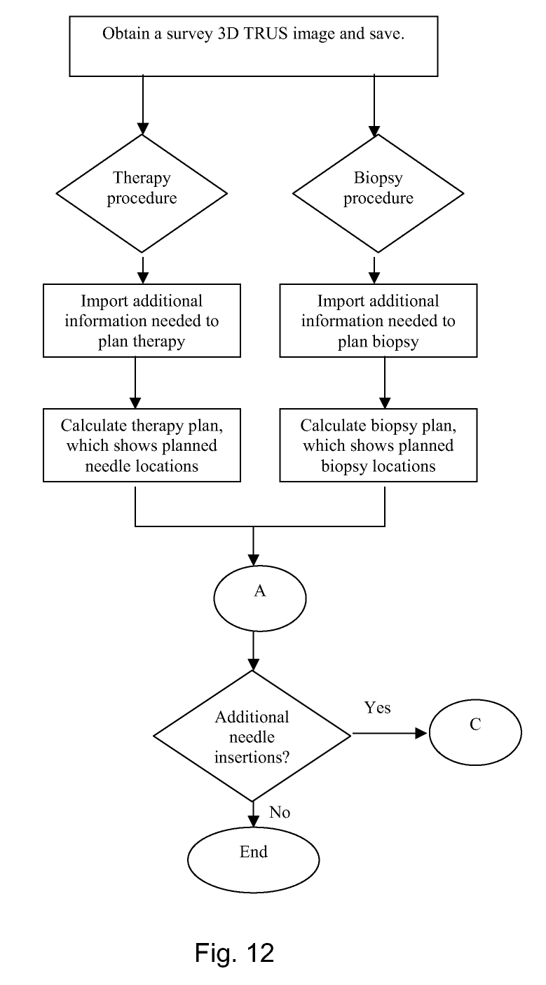

During a prostate brachytherapy procedure, the ultrasound transducer 10 with internal rotation motor assembly is mounted on the base shaft 8 and inserted into the rectum, as is conventionally known. The surgeon, a physician or another healthcare professional then acquires a pre-therapy or pre-biopsy 3D TRUS image by rotating the transducer about its longitudinal axis, while 2D ultrasound images are digitally captured by the computer and reconstructed into a 3D image, as previously described. The 3D TRUS image is then viewed by the person acquiring the image and optionally saved to computer memory, as shown in FIG. 12. The 3D TRUS image can then be recalled at any time, for example to plan the surgical procedure using therapy or biopsy planning software, during the procedure, or afterwards in comparison with post-operative images.

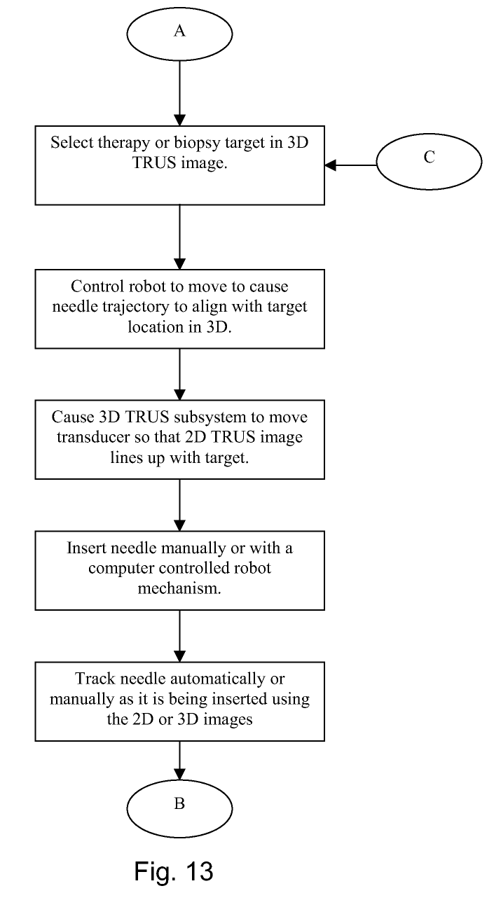

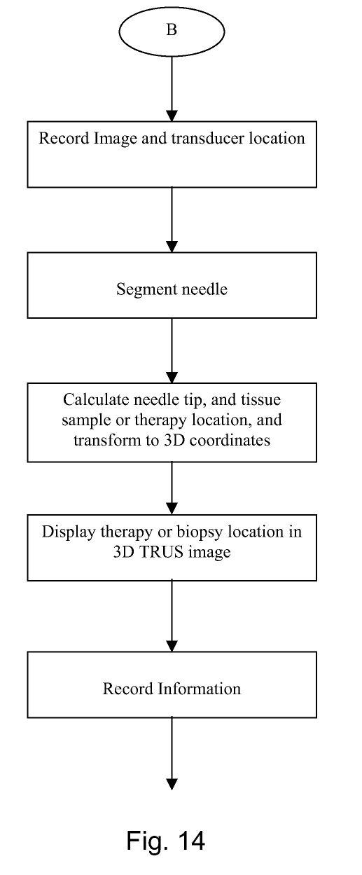

Referring to FIG. 13, after the therapy or biopsy procedure is planned, the robot-aided therapy or biopsy procedure progresses as shown in FIGS. 13 and 14. The surgeon selects a target (using the dose plan or biopsy plan) in the 3D TRUS image of the prostate. The 3D co-ordinates of the target are determined based upon a calibration between the coordinate systems of the apparatus and the 3D TRUS image. A path is then calculated for needle insertion that will reach the desired target. The path may be a straight line along a single vector or a compound path comprising multiple vectors; the latter is particularly useful in cases where interference from the pubic arch prevents direct access to the target. The computer then instructs the motors 9 to move the guide points a and b to the corresponding locations on their respective planes so that the needle will be inserted along the desired path. In addition, the computer may cause the ultrasound transducer 10 to rotate so that the needle can be viewed more clearly in the 2D ultrasound images as the needle is inserted. Alternatively, if the needle is to be inserted into the prostate on an oblique trajectory with respect to the ultrasound transducer axis, then continuous 3D TRUS images are acquired of the region through which the needle is inserted. In both situations, automated needle segmentation is performed to provide the surgeon with real-time or near-real time information on the trajectory of the needle during the procedure.

After the needle has been inserted into the prostate to the satisfaction of the surgeon (either manually, with automated assistance, or a combination thereof), the therapy is delivered through the needle or the lesion is biopsied. In the latter, a device such as a spring-loaded biopsy needle may be used. As shown in FIG. 2, the image of the needle in the prostate can be recorded immediately while the therapy or biopsy is being delivered, and/or afterward. In a preferred embodiment, the computer automatically finds, records and displays the location of the needle tip on the 3D image as an aid to the surgeon.

As shown in FIGS. 12 and 14, if another needle insertion is required to deliver therapy or biopsy to another location, the procedure is repeated. This process is repeated until the therapy or biopsy procedure is completed.

The foregoing describes preferred embodiments of the invention and is not to be construed in a limiting sense. Variants or mechanical equivalents to the way in which the invention works will be apparent to those skilled in the art, along with further features and sub-combinations, and are intended to be encompassed by the following claims.

1) An apparatus for the three-dimensional positioning of a guide for manual insertion of a medical tool within a body, the apparatus comprising:
a) an alignment axis;
b) a guide axis aligned with the guide;
c) a first plane orthogonal to the alignment axis;
d) a second plane orthogonal to the alignment axis and parallel to the first plane, the second plane spaced apart from the first plane along the alignment axis;
e) a first positioning means for positioning a first guide point on the first plane, the guide axis passing through the first guide point;
f) a second positioning means for positioning a second guide point on the second plane, the guide axis passing through the second guide point; and,
g) the first and second positioning means separately adjustable in order to provide a pre-determined angular relationship between the guide axis and the alignment axis.

2) The apparatus according to claim 1, wherein the first and/or second positioning means are manually, automatically, or both manually and automatically adjustable.

3) The apparatus according to claim 1, wherein the first positioning means comprises a means of adjusting the distance between the first guide point and the alignment axis and a means of adjusting the polar position of the first guide point on the first plane by rotating a first gear about a first rotation axis parallel to or collinear with the alignment axis.

4) The apparatus according to claim 3, wherein the means of adjusting the distance between the first guide point and the alignment axis comprises:
a) a first link member having a first end pivotally attached to a first crank member of the first gear and having a second end;
b) a second gear spaced apart from the first gear along the alignment axis and rotatable about a second rotation axis parallel to or collinear with the alignment axis, the second gear having a second crank member;
c) a second link member having a first end and a second end, the first end of the second link member pivotally attached to the second end of the first link member, the second link member pivotally attached between its first and second ends to the second crank member at a link pivot point; and,
d) the first and second gears rotatable to adjust the distance between the first guide point and the alignment axis.

5) The apparatus according to claim 4, wherein the first link member and the second crank member are parallel and wherein the first crank member and second link member are parallel, thereby forming a parallelogram linkage, and wherein the first and second rotation axes are collinear.

6) The apparatus according to claim 4, wherein the first positioning means comprises a first locking means to selectively prevent movement of the second link member relative to the first link member about the link pivot point.

7) The apparatus according to claim 6, wherein the first positioning means comprises a second locking means to selectively prevent movement of at least the first or second gear about its respective rotation axis.

8) The apparatus according to claim 7, wherein the second positioning means is identical to the first positioning means.

9) The apparatus according to claim 4, wherein the second end of the second link member comprises a universal joint or a spherical joint for connecting the guide means with the first link member.

10) The apparatus according to claim 1, wherein the guide telescopes between the first and second guide points.

11) The apparatus according to claim 10, wherein the guide is lockable to selectively prevent telescoping.

12) The apparatus according to claim 1, wherein the guide includes release means operable to release the tool from the guide without adjusting the position of the tool or the apparatus.

13) The apparatus according to claim 1, wherein the tool is a needle and wherein the needle passes through the guide means along the guide axis.

14) The apparatus according to claim 1, wherein the angular relationship between the guide axis and the alignment means is determined with reference to a medical image of an interior of the body.

15) The apparatus according to claim 1, wherein the apparatus further comprises at least one motor connected to the first positioning means and a computer interconnection means for use in automatically adjusting the position of the guide using the motor.

16) The apparatus according to claim 15, wherein the apparatus comprises two motors connected to the first positioning means and two motors connected to the second positioning means.

17) An apparatus for the three-dimensional positioning of a guide for manual insertion of a medical tool within a body, the apparatus comprising:
a) at least one positioning means attached to the guide;
b) at least one motor connected to each positioning means, the motor comprising:
i) an armature;
ii) a gear shaft connected with the armature through a set of enmeshed gears;
iii) a manual adjustment knob connected with the gear shaft;
iv) a slip clutch connecting the gear shaft and the positioning means; and,
c) the manual adjustment knob rotatable to manually adjust the positioning means while the motor is in operation without overdriving the motor.

18) The apparatus of claim 17, wherein the positioning means is both manually and automatically adjustable.

19) The apparatus of claim 17, wherein the set of enmeshed gears comprises a differential drive train having a gear ratio greater than one.

20) The apparatus of claim 17, wherein the apparatus comprises a position encoder for determining the rotational position of the gear shaft.

21) A method of positioning a guide for manual insertion of a medical tool within a body, the method comprising:
a) obtaining a medical image of an interior of the body;
b) providing the medical image to a computer in a digital form;
c) determining with the computer a desired angle for manual insertion of the tool based upon analysis of the image; and,
d) automatically positioning the guide at the desired angle using instructions provided by the computer to a motorized guide positioning apparatus.

22) The method according to claim 21, wherein the method further comprises manually positioning the guide proximal the desired angle prior to automatically positioning the guide.

23) The method according to claim 21, wherein the method further comprises manually adjusting the position of the guide while automatically positioning the guide.

24) The method according to claim 21, wherein the tool is installed within the guide and wherein the method further comprises manually releasing the tool from the guide without adjusting the position of the tool or the guide.

25) The method according to claim 21, wherein the medical image comprises a three-dimensional ultrasound image.
